(19) United States
(12) Patent Application Publication
(10) Pub. No.: US 2024/0247031 A1
(43) Pub. Date: Jul. 25, 2024

(54) COMPOSITION FOR TREATING CANCER CONTAINING TELOMERASE-TARGETING GNRH ANTAGONIST-DERIVED PEPTIDE HS1002

(71) Applicant: Research & Business Foundation Sungkyunkwan University, Suwon-si (KR)

(72) Inventors: Hyung Sik KIM, Anyang-si (KR); Jae Hyeon PARK, Suwon-si (KR); Sang Jeon CHUNG, Suwon-si (KR)

(73) Assignee: Research & Business Foundation Sungkyunkwan University, Suwon-si (KR)

(21) Appl. No.: 18/417,734

(22) Filed: Jan. 19, 2024

(30) Foreign Application Priority Data
Jan. 20, 2023 (KR) 10-2023-0008885

(51) Int. Cl. C07K 7/08; A61K 38/00; A61P 35/00

(52) U.S. Cl. CPC C07K 7/08; A61P 35/00; A61K 38/00

(57) ABSTRACT

Disclosed is a method for preventing or treating cancer by using GnRH and telomerase antagonist-derived peptide HS1002, wherein the present polypeptide can be used to effectively treat cancer and is effective in cancer having a high TERT expression or high telomerase activity, especially, prostate cancer.

### CROSS REFERENCE TO RELATED APPLICATION

This application claims priority and the benefit of Korean Patent Application No. 10-2023-0008885 filed in the Korean Intellectual Property Office on Jan. 20, 2023, the entire disclosure of which is incorporated herein by reference.

### INCORPORATION-BY-REFERENCE OF MATERIAL SUBMITTED BY U.S.P.T.O. EFS-WEB

The instant application contains a Sequence Listing which is being submitted in computer readable form via the United States Patent and Trademark Office eFS-WEB system and which is hereby incorporated by reference in its entirety for all purposes. The XML file submitted herewith contains a 9.58 KB file (NewApp_0421930010_SequenceListing).

### BACKGROUND OF THE INVENTION

### 1. Field of the Invention

The present disclosure was made with the support of the Ministry of Science and ICT, Republic of Korea, under project identification No. project No. 2018M3A9C8021792, which was conducted in the research project named “Development of New Peptide Drug for Prostate Cancer Therapy Using Dual Mechanism(s)” in the research program titled “Bio-Medical Technology Development”, by Sungkyunkwan University, under management of the National Research Foundation of Korea, from 1 Jan. 2022 to 31 Dec. 2022.

The present disclosure relates to a method for preventing or treating cancer by using telomerase-targeting GnRH antagonist-derived peptide.

### 2. Description of the Prior Art

Prostate cancer corresponds to “aggressive cancer” in that 96% of all patients are elderly men over the age of 60 and approximately 20% of patients are diagnosed with metastatic prostate cancer at the time of initial diagnosis. In particular, the number of prostate cancer patients increases every year, but the development of treatments is challenging. Considering the increasing trend of prostate cancer patients, many experts are of the opinion that the prostate cancer treatment market has a high growth potential in the future.

For the treatment of prostate cancer, GnRH receptor agonists have been developed, but caused a side effect called “testosterone surge” or “flare reaction”, and this leads to the development of GnRH receptor (GnRH-R) antagonists. GnRH receptor antagonist peptides competitively bind to GnRH receptors to block the GnRH receptors, and rapidly reduce LH and follicular stimulating hormone (FSH) to reduce the production of testosterone without initial stimulation/surge. However, GnRH receptor antagonist peptides also usually cause side effects, such as swelling and itching, due to histamine secretion. Hence, there is a need to develop enhanced peptide drugs for treatment of prostate cancer.

### SUMMARY OF THE INVENTION

The present inventors have made intensive research efforts to develop peptides having similar sequences to a GnRH antagonist and being capable of effectively treating prostate cancer while minimizing side effects. Consequently, the present inventors have established that HS1002 peptide comprising the amino acid sequence of SEQ ID NO: 2 can reduce the expression of telomerase reverse transcriptase (TERT) and activity of telomerase, leading to an outstanding prostate cancer treatment effect, and thus have completed the present disclosure.

An aspect of the present disclosure is to provide a polypeptide comprising the amino acid sequence of SEQ ID NO: 2.

Another aspect of the present disclosure is to provide a nucleic acid molecule comprising a nucleotide sequence encoding the polypeptide.

Still another aspect of the present disclosure is to provide a recombinant vector comprising the nucleic acid molecule.

Still another aspect of the present disclosure is to provide a pharmaceutical composition comprising a polypeptide comprising the amino acid sequence of SEQ ID NO: 2.

Still another aspect of the present disclosure is to provide a method for prevention or treatment of cancer, the method comprising administering to a subject a pharmaceutical composition comprising a polypeptide comprising the amino acid sequence of SEQ ID NO: 2.

In accordance with an aspect of the present disclosure, there is provided a polypeptide comprising the amino acid sequence of SEQ ID NO: 2.

The present inventors have made intensive research efforts to develop peptides having similar sequences to a GnRH antagonist and being capable of effectively treating prostate cancer while minimizing side effects. Consequently, the present inventors have established that HS1002 peptide comprising the amino acid sequence of SEQ ID NO: 2 can reduce the expression of telomerase reverse transcriptase (TERT) and activity of telomerase, leading to an outstanding prostate cancer treatment effect.

Herein, the polypeptide comprising the amino acid sequence of SEQ ID NO: 2 is named HS1002 peptide. The amino acid sequence of SEQ ID NO: 2 is composed of 11 amino acids.

Herein, the polypeptide may include at least one additional amino acid at the C-terminus and/or N-terminus thereof. The additional amino acid residue may be separately or collectively added for the purpose of improving, for example, productivity, purification, in vivo or in vitro stabilization, coupling with a complex, or detection. By way of example, the polypeptide may further include a cysteine residue at the C-terminus and/or N-terminus thereof. The additional amino acid residue may provide a “tag” for purification or polypeptide detection, and, for example, the tag may be for interaction with an antibody specific therefor. In this regard, His₆ tag, (HisGlu)₃ tag (“HEHEHE” tag), “myc” (c-myc) tag, or “FLAG” tag may be provided for immobilized metal affinity chromatography (IMAC).

The polypeptide comprising the amino acid sequence of SEQ ID NO: 2 of the present disclosure is construed to include not only the amino acid sequence of SEQ ID NO: 2 but also a sequence showing substantial identity to the sequence of SEQ ID NO: 2. The substantial identity means preferably at least 80% homology, more preferably at least 85% homology, still more preferably at least 90% homology, most preferably at least 95% homology in sequences when the present nucleotide sequence is aligned with any other sequence so as to match each other as much as possible and the aligned sequences are analyzed using an algorithm commonly used in the art. Alignment methods for sequence comparison are known in the art. Various methods and algorithms for the alignment are disclosed in Smith and Waterman, Adv. Appl. Math. 2:482(1981); Needleman and Wunsch, J. Mol. Bio. 48:443(1970); Pearson and Lipman, Methods in Mol. Biol. 24: 307-31(1988); Higgins and Sharp, Gene 73:237-44(1988); Higgins and Sharp, CABIOS 5:151-3(1989); Corpet et al., Nuc. Acids Res. 16:10881-90(1988); and Huang et al., Comp. Appl. BioSci. 8:155-65(1992); and Pearson et al., Meth. Mol. Biol. 24:307-31(1994). The NCBI Basic Local Alignment Search Tool (BLAST) (Altschul et al., J. Mol. Biol. 215:403-10(1990)) is accessible from the National Center for Biotechnology Information (NCBI) and, on the Internet, may be used in connection with sequence analysis programs, such as blastp, blastn, blastx, tblastn and tblastx.

A biological functional equivalent, which is an amino acid sequence variant exhibiting biological activity equivalent to that of the polypeptide comprising the amino acid sequence of SEQ ID NO: 2 of the present disclosure may also be used as a polypeptide used in the present disclosure. Such amino acid variations are made based on relative similarity of amino acid side chain substituents, such as hydrophobicity, hydrophilicity, charge, and size. An analysis of sizes, shapes, and types of amino acid side chain substituents can reveal that arginine, lysine, and histidine are all positively charged residues; alanine, glycine, and serine have similar sizes; and phenylalanine, tryptophan, and tyrosine have similar shapes. Thus, based on these considerations, arginine, lysine, and histidine; alanine, glycine, and serine; and phenylalanine, tryptophan, and tyrosine may be considered biologically functional equivalents.

In the introduction of variations, the hydropathy index of amino acids may be considered. Each amino acid has been assigned a hydropathy index on the basis of its hydrophobicity and charge characteristics: isoleucine (+4.5); valine (+4.2): leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5): aspartate (−3.5); asparagine (−3.5); lysine (−3.9); and arginine (−4.5). The hydrophobic amino acid index is very important in conferring interactive biological function on a protein. It is known that certain amino acids may be substituted for other amino acids having a similar hydropathy index or score, and still retain a similar biological activity. In the introduction of variations based upon the hydropathy index, the substitution is made between amino acids having a difference in the hydrophobic index within preferably ±2, more preferably ±1, and still more preferably ±0.5.

Meanwhile, it is also well known that substitutions between amino acids having similar hydrophilicity values result in proteins with equivalent biological activity. As disclosed in U.S. Pat. No. 4,554,101, each amino acid residue has been assigned the following hydrophilicity values: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In the introduction of variations based upon the hydrophilicity value, the substitution is made between amino acids having a difference in the hydrophilicity value within preferably ±2, more preferably ±1, and still more preferably ±0.5.

Amino acid exchanges in proteins that do not entirely alter the activity of the molecule are known in the art (H. Neurath, R. L. Hill, The Proteins, Academic Press, New York, 1979). The most common exchanges occur between amino acid residues Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu, and Asp/Gly.

In an embodiment of the present disclosure, the polypeptide is composed of the amino acid sequence of SEQ ID NO: 2.

In an embodiment of the present disclosure, the polypeptide comprising the amino acid sequence of SEQ ID NO: 2 inhibits the proliferation and growth of cancer cells.

In an embodiment of the present disclosure, the polypeptide comprising the amino acid sequence of SEQ ID NO: 2 has characteristics of inhibiting the expression of telomerase reverse transcriptase (TERT), inhibiting the activity of telomerase, or a combination thereof. The polypeptide of the present disclosure may inhibit the expression of telomerase reverse transcriptase or the activity of telomerase by, for example, 10%, 20%, 30%, 40%, 50%, 60%, 70%, 80%, or 90% compared with the control group not treated with the polypeptide, but is not limited thereto.

In an embodiment of the present disclosure, the polypeptide comprising the amino acid sequence of SEQ ID NO: 2 reduces the expression and transcriptional activity of c-Myc. Alternatively, the polypeptide inhibits the activation of AKT, ERK1/2, and mTOR pathways in cancer cells. The polypeptide of the present disclosure may inhibit the expression and transcriptional activity of c-Myc, the expression of telomerase reverse transcriptase, or the activity of telomerase by, for example, 10%, 20%, 30%, 40%, 50%, 60%, 70%, 80%, or 90% compared with the control group not treated with the polypeptide, but is not limited thereto.

As used herein, the term telomere denotes a chromomere located at the end of a chromosome. Typically, telomeres consist of “TTAGGG” repeats in which non-coding G is repeated (about 1,500 to 2,500 times), and this region diminishes with each cell division without storing genetic information. Telomeres gradually shorten in length with each cell division and thus determine the lifespan of cells. However, cancer cells can continuously divide due to their immortality, and telomerase that polymerizes telomeres to extend their length is overexpressed in the cancer cells. Cancer cells, evading apoptosis, maintain a constant length of telomeres, unusually shorter than normal cells, rather than indefinitely extending telomeres using this enzyme. A study has suggested that these characteristics contribute to the genetic instability that cancer cells increase diversity, but a clear correlation has not been revealed.

As used herein, the term telomerase reverse transcriptase (TERT) refers to the catalytic subunit of the telomerase enzyme, which constitutes the most important unit of the telomerase complex, together with the telomerase RNA component. TERT is responsible for catalyzing the addition of nucleotides of the TTAGGG sequence at the end of each of the chromosome telomeres.

As used herein, the term “expression” refers to the expression of a protein or the expression of a gene encoding the same, and the “expression of a gene” may refer to the transcription of the gene into a polynucleotide, the translation into a polypeptide, or the modification of a polynucleotide and/or a polypeptide (e.g., including post-translational modification of a polypeptide). As used herein, the term “expressed gene” encompasses a gene that is transcribed as mRNA and then translated into a polypeptide, or a gene that is transcribed into RNA but not translated into a polypeptide (e.g., tRNA and rRNA).

The wording “inhibiting the expression of a gene or protein” denotes a measurable decrease in the quantitative value of expression of a gene or protein in a test group compared with a control group. For example, the wording may denote that the expression level of a gene or protein in a test group is 90% or less, 80% or less, or 70% or less compared with a control group, but is not limited thereto.

In an embodiment of the present disclosure, the polypeptide binds to a gonadotropin-releasing hormone (GnRH) receptor.

Gonadotropin-releasing hormone (GnRH) is a releasing hormone (RH) at an early stage that is released from the hypothalamus to induce the secretion of gonadotropins in the anterior pituitary, thereby inducing the release of follicle-stimulating hormone (FSH) and luteinizing hormone (LH). GnRH receptor agonists or GnRH receptor antagonists that bind to GnRH receptors may be used in the treatment of prostate cancer.

In an embodiment of the present disclosure, the polypeptide comprising the amino acid sequence of SEQ ID NO: 2 has characteristics of inhibiting the secretion of testosterone, reducing the weight of the seminal vesicles, or a combination thereof.

In accordance with an aspect of the present disclosure, there is provided a nucleic acid molecule comprising a nucleotide sequence encoding a polypeptide comprising the amino acid sequence of SEQ ID NO: 2.

As used herein, the term “nucleic acid molecule” comprehensively encompasses DNA (gDNA and cDNA) and RNA molecules, and the nucleotide, which is a basic constituent of the nucleic acid molecule, include not only natural nucleotides but also analogues having modified sugar or base moieties (Scheit, Nucleotide Analogs, John Wiley, New York (1980); and Uhlman and Peyman, Chemical Reviews, 90:543-584(1990)).

The nucleotide sequence encoding the polypeptide of the present disclosure may be any nucleotide sequence that encodes the amino acid sequence of the peptide HS1002, and it would be obvious to a person skilled in the art that the nucleotide of the present disclosure is not limited to any specific nucleotide sequence.

The reason is that although the nucleotide sequence undergoes variation, the expression of the nucleotide sequence variant into a protein may not cause a change in the protein sequence. This is called codon degeneracy. Therefore, the nucleotide sequence includes nucleotide sequences containing functionally equivalent codons, codons encoding the same amino acid (e.g., the number of codons for arginine or serine is six due to codon degeneracy), or codons encoding biologically equivalent amino acids.

According to a specific embodiment of the present disclosure, the sequences of polypeptides comprising the amino acid sequence of the peptide HS1002 of the present disclosure are included in the sequence listing attached hereto.

A nucleic acid molecule comprising a nucleotide sequence encoding a polypeptide comprising the amino acid sequence of the peptide HS1002 is also construed to include nucleotide sequences showing substantial identity to the nucleotide sequence. The substantial identity denotes at least 80% homology, more preferably at least 90% homology, most preferably at least 95%, 97%, 98%, or 99% homology in nucleotide sequences when the present nucleotide sequence is aligned with any other sequence so as to match each other as much as possible and the aligned sequences are analyzed using an algorithm commonly used in the art.

Considering the above-described variants having biological equivalent activity, the nucleic acid molecule comprising a nucleotide sequence encoding a polypeptide comprising the amino acid sequence of the peptide HS1002 of the present disclosure is construed to also include sequences having substantial identity to the sequences described in the sequence listing. The substantial identity denotes at least 61% homology, more preferably 70% homology, still more preferably 80% homology, most preferably 90% homology in sequences when the present nucleotide sequence is aligned with any other sequence so as to match each other as much as possible and the aligned sequences are analyzed using an algorithm commonly used in the art.

In accordance with an aspect of the present disclosure, there is provided a recombinant vector comprising the nucleic acid molecule.

As used herein, the term “vector” refers to any means for expressing a target gene in a host cell, and encompasses: plasmid vectors; cosmid vectors; and viral vectors, such as bacteriophage vectors, adenoviral vectors, retroviral vectors, and adeno-associated viral vectors.

According to a specific embodiment of the present disclosure, the nucleic acid molecule comprising the nucleotide sequence encoding the polypeptide comprising the amino acid sequence of the peptide HS1002 is operatively linked to a promoter of the vector.

As used herein, the term “operatively linked” refers to a functional linkage between a nucleic acid expression control sequence (e.g., a promoter, a signal sequence, or an array of transcription regulation factor binding sites) and another nucleic acid sequence, whereby the control sequence controls the transcription and/or translation of the another nucleic acid sequence.

The recombinant vector system of the present disclosure can be constructed by various methods known in the art, and a specific method therefor is disclosed in Sambrook et al., Molecular Cloning: A Laboratory Manual, Cold Spring Harbor Laboratory Press (2001), which is incorporated herein by reference.

The vector of the present disclosure may be typically constructed as a vector for gene cloning or a vector for protein expression. In addition, the vector of the present disclosure may be constructed by using a prokaryotic or eukaryotic cell as a host.

For example, in cases where the vector of the present disclosure is an expression vector and an eukaryotic cell is used as a host cell, a promoter derived from the genome of a mammalian cell (e.g., metallothionein promoter, beta-actin promoter, human hemoglobin promoter, and human muscle creatine promoter) or a promoter derived from mammalian viruses (e.g., adenovirus late promoter, vaccinia virus 7.5 K promoter, SV40 promoter, cytomegalovirus promoter, tk promoter of HSV, mouse mammary tumor virus (MMTV) promoter, LTR promoter of HIV, promoter of Moloney virus, Epstein-Barr virus (EBV), and Rous sarcoma virus (RSV)) may be used, and these typically have a polyadenylated sequence as the transcription termination sequence.

The vector of the present disclosure may be fused with the other sequences to facilitate the purification of polypeptides or proteins expressed therefrom. Examples of the fusion sequence include glutathione S-transferase (Pharmacia, USA), maltose-binding proteins (NEB, USA), FLAG (IBI, USA), 6-(hexahistidine; Quiagen, USA), and the like.

Meanwhile, the expression vector of the present disclosure includes, as a selective marker, an antibiotic agent-resistant gene that is commonly used in the art, and examples thereof include resistant genes against ampicillin, gentamycin, carbenicillin, chloramphenicol, streptomycin, kanamycin, geneticin, neomycin, and tetracycline.

In accordance with an aspect of the present disclosure, there is provided a host cell transformed with the recombinant vector.

Host cells capable of stably and continuously cloning and expressing the vector of the present disclosure may be known in the art, and any host cell may be used. Examples of eukaryotic host cells suitable for the vector may include monkey kidney cells (COS7), NSO cells, SP2/0, Chinese hamster ovary (CHO) cells, W138, baby hamster kidney (BHK) cells, MDCK, myeloma cell lines, HuT 78 cells, and HEK-293 cells, but are not limited thereto.

As used herein, the term “transformed”, “transduced”, or “transinfected” refers to a process for delivering or introducing an exogenous nucleic acid into a host cell. A “transformed”, “transduced”, or “transfected” cell is a cell that has been transformed, transduced, or transfected with an exogenous nucleic acid, and the cell includes the primary subject cell and its progenies resulting from passages.

In accordance with an aspect of the present disclosure, there is provided a pharmaceutical composition comprising a polypeptide comprising the amino acid sequence of SEQ ID NO: 2.

The pharmaceutical composition of the present disclosure may be formulated by using a pharmaceutically acceptable carrier and/or excipient according to a method that can be easily performed by a person skilled in the art to which the present disclosure pertains, and thus the pharmaceutical composition may be provided as a unit dosage form or may be packed in a multi-dose container. Particularly, the formulation may be variously prepared as a medicine for internal use, an injection, a gel, a film, a perfusion, a spray liquid, a liquid preparation for spray or vaporization administration, a bubble aerosol preparation, an infusion preparation, and the like, and may be in the form of a solution, suspension, or emulsion in an oil or water medium, an extract, a pulvis, a suppository, a powder, granules, a tablet, or a capsule. In addition, the formulation may additionally contain a dispersant or a stabilizer.

If the formulation of the pharmaceutical composition is a gel or film, the pharmaceutical composition covers or is applied to a target site during or after surgery.

If the formulation of the pharmaceutical composition is a perfusion, the pharmaceutical composition is perfused to a target site during or after surgery.

If the formulation of the pharmaceutical composition is a spray liquid, the pharmaceutical composition is widely and uniformly supplied to a target site or covers a target site to an arbitrary thickness during surgery.

If the formulation of the pharmaceutical composition is a liquid preparation for spray or vaporization administration, the pharmaceutical composition is widely and uniformly sprayed onto a target site in a mist state.

If the formulation of the pharmaceutical composition is a bubble aerosol preparation, the pharmaceutical composition is widely and uniformly supplied to a target site or covers a target site to an arbitrary thickness during surgery.

If the formulation of the pharmaceutical composition is an infusion preparation, the pharmaceutical composition is administered by a method such as instillation, before, during, or after surgery.

The pharmaceutical composition of the present disclosure may further contain an appropriate carrier, excipient, or diluent that is commonly used in the preparation of a pharmaceutical composition. Examples of the carrier, excipient, and diluent include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and a mineral oil.

In an embodiment of the present disclosure, the pharmaceutical composition has reduced side effects compared with existing anticancer drugs. In the pharmaceutical composition of the present disclosure, the peptide as an active ingredient, unlike compound-based anticancer drugs, has no toxicity by metabolites, resulting in relatively few side effects. In an embodiment of the present disclosure, no change in mouse body weight was observed when the pharmaceutical composition of the present disclosure was administered to mice, and this may indicate that the composition of the present disclosure has few side effects.

In an embodiment of the present disclosure, the pharmaceutical composition activates an immune response of a subject. The activating of an immune response may be activating immune cells.

As used herein, the term “activating immune cells” may denote, for example, the successful introduction of an active substance into immune cells, the priming of antigen-presenting cells, polarization of macrophages, the secretion of cytokines, chemokines, or granzymes of immune cells, the induction of immune cell death (ICD) of tumor cells by immune cells, and the inhibition of tumor microenvironments, but is not limited thereto. More specifically, the cytokines may be interferons. As used herein, the term “interferons (IFNs)” refers to cytokines that play an important role in the inflammation, immune regulation, tumor cell recognition, and T cell responses. Interferon-gamma is a specific type of cytokine that is mainly produced by T cells and NK cells. Interferon-gamma plays a role in increasing immune responses, and especially, playing a crucial role in strengthening defenses against pathogens within cells, promoting immune responses within cells, and inhibiting the growth of cancer cells. Interferon-gamma helps to promote or inhibit specific immune responses by sending signals to other components of the immune system. As used herein, the term “granzyme B” denotes an important ceramic protein produced by cytotoxic T cells and NK cells. These cells target cells infected with viruses or having lesions such as cancer cells. Granzyme B is injected into these target cells to degrade proteins inside the cells, inducing cell death. Through this process, granzyme B plays an important role in the immune system eliminating cells with lesions. The polypeptide comprising the amino acid sequence of SEQ ID NO: 2 may increase the secretion of interferon-gamma or granzyme B by at least 10%, 20%, 30%, 40%, 50%, 60%, 70%, 80%, 90%, 100%, 150%, 200%, 250%, or 300%, but is not limited thereto.

In an embodiment of the present disclosure, the pharmaceutical composition may further contain an immunotherapeutic agent of: immune checkpoint inhibitors (e.g., pembrolizumab, nivolumab, ipilimumab, etc.) that block the ability of cancer cells to evade the immune system while targeting immune checkpoints, such as PD-1, PD-L1, and CTLA-4; cell therapeutic agents (e.g., Kymriah, Yescarta, etc.) that modify cancer cells collected from patients to allow the immune cells to recognize specific cancer cells and then again inject the modified cancer cells into the patients; cancer vaccines (e.g., Provenge, etc.) that help the immune system more effectively recognize and attack cancer cells; or a combination thereof.

In an embodiment of the present disclosure, the pharmaceutical composition further contains an immune-enhancing substance or adjuvant. More specifically, the immune enhancer (adjuvant) generally denotes any substance (e.g., alum, Freund's complete adjuvant, Freund's incomplete adjuvant, LPS, poly IC, poly AU, etc.) that increases body fluids or cellular immune responses against antigens. Examples of the immune enhancers of the present disclosure include alum, Complete Freund's Adjuvant, Incomplete Freund's Adjuvant, LPS, poly IC, poly AU, Lipofectin, Lipotaxi, CaPO₄, DEAE dextran, Polybrene, saponin, an inorganic material such as aluminum hydroxides, gel, lysolecithin, pluronic polyols, polyanions, peptides, a surfactant such as an oil or hydrocarbon emulsion, dinitrophenol, an aluminum salt (aluminum hydroxide or aluminum phosphate), MF59, AS01, AS02, AS03, AS04, or CpG DNA, but are not limited thereto.

In accordance with an aspect of the present disclosure, there is provided a method for preventing or treating cancer, the method comprising administering to a subject a pharmaceutical composition comprising a polypeptide comprising the amino acid sequence of SEQ ID NO: 2.

As used herein, the term “prevention” denotes the preventive or protective treatment of a disease or a disease condition. As used herein, the term “treatment” denotes the alleviation, suppression, amelioration, or eradication of a disease condition. For example, the term denotes the minimization of diffusion or deterioration of a disease by administration of a therapeutic agent to a subject.

As used herein, the term “subject” or “patient” refers to an animal including a human, pig, chimpanzee, dog, cat, cow, mouse, rabbit or rat, and preferably a mammal.

The pharmaceutical composition according to the present disclosure is administered at a pharmaceutically effective amount. Herein, the pharmaceutically effective amount denotes an amount sufficient to treat a disease at a reasonable benefit/risk ratio applicable to medical treatment, and the effective dosage may be determined depending on factors including the type of disease in a patient, the severity of disease, the activity of a drug, the sensitivity to a drug, the time of administration, the route of administration, the rate of excretion, the treatment period, and a drug used in combination, and other factors well known in the medical field. The amount of the composition used may vary depending on the age, sex, and weight of a patient, but a sufficient amount of the composition may be administered once to several times per day so that the concentration of the peptide in blood is appropriate for treating cancer.

The dosage of the composition may be decreased or increased depending on the route of administration, the severity of disease, sex, weight, age, and the like. Therefore, the above-described dosage is not intended to restrict the range of the present disclosure in any way.

The dosage of the pharmaceutical composition of the present disclosure is preferably 0.001 to 100 mg/kg (weight) per day, and the above-described dosage of the pharmaceutical composition may be topically to a region desired to be applied, depending on the purpose of application.

The pharmaceutical composition of the present disclosure may be administered to a subject through various routes. All modes of administration may be considered, and for example, the pharmaceutical composition may be administered by intracerebral administration, oral intake, subcutaneous injection, intraperitoneal administration, intravenous injection, intramuscular injection, intrathecal administration, sublingual administration, buccal mucosal administration, rectal insertion, vaginal insertion, ocular administration, auricular administration, nasal administration, inhalation, nebulization through the mouth or nose, dermal administration, transdermal administration, or the like. Preferably, the pharmaceutical composition may be administered by intravenous injection.

The pharmaceutical composition according to the present disclosure may be administered as an individual therapeutic agent or administered in combination with another therapeutic agent, and may be administered sequentially or simultaneously with a conventional therapeutic agent, and may be administered in either single or multiple doses. It is important to administer the pharmaceutical composition at the minimum amount that can obtain the maximum effect without causing side effects, considering all the above-described factors, and such an amount can be easily determined by a person skilled in the art to which the present disclosure pertains.

In an embodiment of the present disclosure, the cancer is associated with an increase in telomerase reverse transcriptase expression, an increase in telomerase activity, or a combination thereof.

In an embodiment of the present disclosure, the method for preventing or treating cancer is activating an immune response to cancer cells.

In an embodiment of the present disclosure, the method for preventing or treating cancer further includes administering an immunotherapeutic agent.

Examples of the immunotherapeutic agent may include: immune checkpoint inhibitors (e.g., pembrolizumab, nivolumab, ipilimumab, etc.) that block the ability of cancer cells to evade the immune system while targeting immune checkpoints, such as PD-1, PD-L1, and CTLA-4; cell therapeutic agents (e.g., Kymriah, Yescarta, etc.) that modify cancer cells collected from patients to allow the immune cells to recognize specific cancer cells and then again inject the modified cancer cells into the patients; cancer vaccines (e.g., Provenge, etc.) that help the immune system more effectively recognize and attack cancer cells; or a combination thereof.

In an embodiment of the present disclosure, the method for preventing or treating cancer further includes administering an immune-enhancing substance or adjuvant.

The immune enhancer (adjuvant) generally denotes any substance (e.g., alum, Freund's complete adjuvant, Freund's incomplete adjuvant, LPS, poly IC, poly AU, etc.) that increases body fluids or cellular immune responses against antigens. Examples of the immune enhancers of the present disclosure include alum, Complete Freund's Adjuvant, Incomplete Freund's Adjuvant, LPS, poly IC, poly AU, Lipofectin, Lipotaxi, CaPO₄, DEAE dextran, Polybrene, saponin, an inorganic material such as aluminum hydroxides, a gel, lysolecithin, pluronic polyols, polyanions, peptides, a surfactant such as an oil or hydrocarbon emulsion, dinitrophenol, an aluminum salt (aluminum hydroxide or aluminum phosphate), MF59, AS01, AS02, AS03, AS04, or CpG DNA, but are not limited thereto.

In an embodiment of the present disclosure, the cancer is selected from the group consisting of prostate cancer, ovarian cancer, or a combination thereof. However, the cancer is not limited thereto, and encompasses any benign and malignant tumors with an increase in the expression of telomerase reverse transcriptase or an increase in the activity of telomerase. Examples of the cancer may include carcinoma, lymphoma, blastoma, sarcoma, neuroendocrine tumor, mesothelioma, schwannoma, meningioma, adenocarcinoma, or melanoma, but are not limited thereto. More specifically, the cancer may be selected from the group consisting of squamous cell carcinoma, lung cancer, peritoneal cancer, liver cell carcinoma, stomach cancer, gastrointestinal cancer, pancreatic cancer, cervical cancer, ovarian cancer, liver cancer, bladder cancer, hepatocellular carcinoma, breast cancer, colon cancer, rectal cancer, kidney cancer, prostate cancer, thyroid cancer, testicular cancer, and esophageal cancer.

Features and advantages of the present disclosure are summarized as follows.
(a) The present disclosure provides a polypeptide comprising the amino acid sequence of SEQ ID NO: 2.
(b) The present disclosure provides a nucleic acid molecule comprising a nucleotide sequence encoding the polypeptide.
(c) The present disclosure provides a recombinant vector comprising the nucleic acid molecule.
(d) The present disclosure provides a pharmaceutical composition comprising a polypeptide comprising the amino acid sequence of SEQ ID NO: 2.
(e) The present disclosure provides a method for preventing or treating cancer, the method comprising administering to a subject a pharmaceutical composition comprising a polypeptide comprising the amino acid sequence of SEQ ID NO: 2.
(f) The polypeptide of the present disclosure can be used to effectively treat cancer and is effective in cancer having a high TERT expression or high telomerase activity, especially, prostate cancer.

### BRIEF DESCRIPTION OF THE DRAWINGS

The above and other aspects, features, and advantages of the present disclosure will be more apparent from the following detailed description taken in conjunction with the accompanying drawings.

FIGS. 1A to 1C show the correlation between the progression of prostate cancer and the level of hTERT expression. FIG. 1A shows the immunohistochemistry results of tumor cells according to the Stage and Gleason grade. FIG. 1B shows the hTERT IHC score according to the Stage, and FIG. 1C shows the hTERT IHC score according to the Gleason grade.

FIGS. 2A to 2C show the hTERT expression levels and telomerase activities in prostate cancer cells (PC3, DU145, LNCaP). FIG. 2A shows the western blot results of hTERT and β-actin in each type of prostate cancer cells, and FIGS. 2B and 2C show the quantification results of hTERT protein expression level and telomerase activity in each type of prostate cancer cell line.

FIG. 3 shows the structural difference between a gonadotropin-releasing hormone (GnRH) receptor ligand and a telomerase-derived peptide (GV1001), to design a peptide targeting hTERT.

FIG. 4 shows amino acid sequences of six synthesized peptides.

FIG. 5 shows amino acid sequences of the telomerase-derived peptide (GV1001) and synthesized peptides (HS1001 and HS1002).

FIG. 6 shows the comparison of cytotoxicity to prostate cancer cells (LNCaP) after treatment with peptides (HS1001 and HS1002).

FIGS. 7A to 7C show the apoptotic and proliferation inhibitory abilities on prostate cancer cells (LNCaP) after treatment with peptides (HS1001 and HS1002). FIG. 7A shows the proliferation inhibitory effect on cancer cells by the treatment with peptides. FIGS. 7B and 7C show the apoptosis promoting effect on cancer cells by the treatment with peptides.

FIG. 8 shows the comparison of cytotoxicity on various types of cancer cell lines (LNCaP, PC3, DU145, MDA-MB-231, SKOV3, and MCF-7) after treatment with the peptide (HS1002).

FIG. 9 shows changes in expression levels of proteins involved in apoptosis after treatment with the peptide (HS1002) at different concentrations.

FIG. 10 shows the hTERT expression levels and telomerase activities in prostate cancer cells (LNCaP) after treatment with the peptides (HS1001 and HS1002).

FIG. 11 shows the hTERT expression levels and telomerase activities in prostate cancer cells (LNCaP) after treatment with the peptide (HS1002) at different concentrations.

FIG. 12 shows that HS1002 targets c-Myc to inhibit hTERT expression. (A) Western blotting analysis of c-Myc and hTERT expression in LNCaP cells after 72 h of treatment with HS1002. The c-Myc inhibitor 10058-F4 was used as a positive control. (B, C) Quantification of c-Myc and hTERT expression was determined by Image J software. (D) Effect of HS1002 treatment on c-Myc transcriptional activity. The values represent the means±S.D.*p<0.05 and **p<0.01 vs. the control group.

FIG. 13 shows that suppression of the hTERT regulation pathway by HS1002. AKT, ERK, and mTOR signaling are involved in hTERT expression and activation. (A) Changes in the expression of signaling proteins (AKT, ERK1/2, and mTOR) after HS1002 treatment in LNCaP cells. The loading control used in this experiment was β-actin. (B-D) Densitometric analysis of the western blot expression using Image J software. The values represent the means±S.D.*p<0.05 and **p<0.01 vs. the control group

FIG. 14 shows the in vivo anti-cancer effect of HS1002 in LNCaP tumors. (A, B) Change in the expression of c-Myc and signaling protein (ERK1/2, AKT) levels in LNCaP-xenografted tumors. The values represent the means±S.D.*p<0.05 vs the vehicle group; #p<0.05 between two groups.

FIG. 15 shows the in vivo administration of HS1002 influences serum testosterone levels. (A) Experimental design of HS1002 administration. (B) Serum testosterone level of vehicle or HS1002 (1 mg/kg)-treated BALB/c mice (n=4). (C) Seminal vesicles and testis were collected from BALB/c mice after 10 days of treatment with vehicle or HS1002 (1 mg/kg). (D) Percentage of seminal vesicles/body weight. The values represent the means±S.D.*p<0.05 and vs. the vehicle group.

FIG. 16 shows the activation of GnRHR signaling by HS1002 in GnRHR-transfected HEK293 cells. (A) Western blot analysis of GnRHR expression in HEK293-Vector and HEK293-GnRHR. The loading control used in this experiment was β-actin. (B) The band intensity was quantified using Image J software. *p<0.05 vs. the vector group. Effect of HS1002 or LA on (C) cytosolic calcium flux change and (D) CRE-luciferase activity in HEK293-Vector and HEK293-GnRHR cells. LA was used as a positive control. The values represent the means±S.D.*p<0.05 and **p<0.01 vs. the control group; #p<0.05 and ##p<0.01 between two groups. Leuprolide acetate; LA

FIGS. 17A to 17F show the tumor volumes and weights after subcutaneous administration of the novel peptide derivative (HS1002) to animals implanted with prostate cancer cells (LNCaP). FIG. 17A shows experimental groups and experimental schedules; FIG. 17B shows the change in tumor volume; FIG. 17C shows tumor weights; FIG. 17D shows the change in mouse weight; and FIGS. 17E and 17F show tumor images.

FIG. 18 shows the hTERT expression level in prostate cancer cells (LNCaP) and proliferation inhibitory ability on the prostate cancer cells after treatment with the peptide (HS1002) at different concentrations.

FIGS. 19A and 19B show the comparison of MC38, 4T1 tumor growth inhibition by HS1002. (A) Experimental scheme of syngeneic mouse tumor model. (B, C) The growth curves of MC38 or 4T1-implanted mouse. (D, E) Tumor weights of tumor bearing mice treated with vehicle, HS1002 (1 mg/kg), or GV1001 (1 mg/kg). The values represent the means±S.D.*p<0.05 and **p<0.01 vs the vehicle group.

FIGS. 20A and 20B show the effect of HS1002 on tumor infiltrating leukocytes in MC38 tumors. (A) Absolute counts of CD45⁺ cells per gram tumor in vehicle or HS1002-treated MC38 tumor. (B, C) Percentages of granzyme B and IFN-γ positive CD8⁺ cell population in MC38 tumor-bearing mice treated with vehicle or HS1002. (D) Heatmap of cluster analysis using differentially expressed genes between vehicle- or HS1002-treated MC38 tumors. (E) Biological process analysis was performed using DAVID to selected upregulated genes (P<0.05, FC>1.5) The values represent the means±S.D.*p<0.05 and **p<0.01 vs the vehicle group.

### DETAILED DESCRIPTION OF THE EXEMPLARY EMBODIMENTS

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

### Example 1: Materials and Methods

### 1-1. Reagents

The peptide was synthesized using solid-phase synthesis methods by Anygen Co., Ltd. (Gwangju, Korea). A peptide solution was prepared in Dulbecco's phosphate-buffered saline (DPBS). Culture medium and fetal bovine serum (FBS) were obtained from Gibco Invitrogen Corporation (Carlsbad, CA, USA). Primary antibodies for c-Myc, AKT, p-AKT (Ser473), ERK1/2, p-ERK1/2 (Thr202/Tyr204), p-mTOR (Ser2448), and mTOR were obtained from Cell Signaling Technology (Beverly, MA, USA). Primary antibodies against GnRHR and hTERT were obtained from Abcam (Cambridge, MA, USA). The primary antibody against β-actin was purchased from Santa Cruz Biotechnology (Santa Cruz, CA, USA). Horseradish peroxidase (HRP)-conjugated secondary antibodies were obtained from Novus Biologicals (Minneapolis, MN, USA). Fluorochrome-conjugated antibodies were used for flow cytometry: PerCP/Cyanin5.5 anti-CD45 (clone 30-F11), FITC anti-CD8a (clone 53-6.7), APC anti-Granzyme B (clone QA16A02), and PE anti-IFN-γ(XMG1.2) from Biolegend (San Diego, CA, USA).

### 1-2. Cell Culture and Cell Lines

Human embryonic kidney cell line (HEK293), and mouse cancer cell lines (MC38, 4T1) were purchased from the American Type Culture Collection (Manassas, VA, USA). All cell lines were grown in Dulbecco's Modified Essential Medium (DMEM) or Roswell Park Memorial Institute (RPMI) 1640 supplemented with 10% FBS, 100 U/mL penicillin, and 100 μg/mL streptomycin (WelGENE, Daegu, South Korea) in a humidified atmosphere of 5% CO₂ at 37° C. To obtain GnRHR-overexpressing HEK293 (HEK293-GnRHR) cells, transfection of the pCMV-AC-GFP-GnRHR vector (HEK293-GnRHR) was performed using Lipofectamine™ 3000 according to the manufacturer's instructions (Cat. 100022052, Thermo Fisher Scientific, Waltham, MA, USA). Colonies of geneticin-resistant cells were selected by adding G418 (800 μg/mL, Sigma) to the culture medium. The HEK293-pCMV-AC-GFP vector (HEK293-Vector) cells were used as a mock-transfection group.

### 1-3. siRNA Transfection

10 nM GnRHR dsiRNA (Cat. hs.Ri.GNRHR.13, IDT, Coralville, IA, USA) using Lipofectamine™ RNAiMAX (Cat. 56532, Thermo Fisher Scientific) was used to knockdown the endogenous levels of GnRHR in LNCaP cells. The universal negative control dsiRNA (Cat. 51-01-14-03, IDT) was used as a negative control.

### 1-4. Western Blot Analysis

The cells and tumor tissues were homogenized using the PRO-PREP™ extraction solution (iNtRON Biotechnology, Seongnam, Korea). After incubation, the homogenate was centrifuged at 16,000×g for 20 min at 4° C., and the supernatant was collected for western blot analysis. Subsequently, an equivalent quantity of protein was separated using sodium dodecyl sulfate polyacrylamide gels and transferred onto a polyvinylidene difluoride (PVDF) membrane. The PVDF membranes were incubated with the primary antibodies overnight at 4° C., washed with tris-buffered saline, and then incubated with HRP-conjugated anti-mouse or anti-rabbit antibodies. The bands were visualized using a ChemiDoc imaging system (Bio-Rad, Hercules, CA, USA). The band intensities were determined using an Image J software.

### 1-5. c-Myc Transcriptional Activity

The c-Myc Transcription Factor Assay Kit (Cat. ab207200, abcam) was used to quantify the activation of c-Myc in nuclear extracts after peptide treatment. In brief, LNCaP cells were subjected to treatment with HS1002 or 10058-F4 for 72 h, followed by detection of c-Myc transcriptional activity according to the manufacturer's instructions. The nuclear extracts were isolated using the nuclear extraction kit (Cat. ab113474, abcam). The protein concentration was quantified using the Bradford method (798 μL Bradford, 200 μL water, and 2 μL of the protein) at a wavelength of 595 nm (SmartSpecPlus; BioRad). For the detection of c-Myc transcriptional activity, 5 μg of the isolated nuclear extracts were used, and the absorbance was measured on a spectrophotometer (Synergy HT; BioTek) at OD 450 nm.

### 1-6. Intracellular Calcium Flux Assay

Intracellular calcium mobilization was measured using the Calcium Flux Assay Kit (Cat. ab233472, abcam) in response to different concentrations of each peptide. The percentage increase was calculated relative to basal fluorescence intensity. The experiment was performed in triplicate.

### 1-7. CRE-Luciferase Reporter Assay

The HEK293-pCMV6-AC-GFP and HEK293-GnRHR cells (4×10⁴ cells/well) were grown in 96-well plates and co-transfected with a luciferase reporter plasmid containing a cAMP response element (CRE) using Lipofectamine 3000 (Life Technologies). The transfected cells were exposed to different concentrations of peptide for 24 h, and the promoter activity was measured by a dual-luciferase reporter assay system (BPS Bioscience). The firefly and Renilla luciferase activities were measured using a microplate luminometer (Thermo, Varioskan LUX Multimode Microplate Reader). The calculation of relative luciferase activities involved the normalization of the firefly luciferase activity driven by the promoter of Renilla luciferase.

### 1-8. Animal Study

All mice were purchased from Central Lab Animal Inc. (Hamamatsu, Japan). They were raised in a 12 h environmental light/dark cycle with controlled temperature (22±2° C.) conditions. The experimental protocol was approved by the Sungkyunkwan University Institutional Animal Care and Use Committee (SKKUIACUC2021-06-27-1, SKKUIACUC2023-01-07-1). For the establishment of tumors, LNCaP (1×10⁷), MC38 (2×10⁵), and 4T1 (1×10⁶) suspended in serum-free media containing 50% Matrigel was subcutaneously administered in the right flank of mice, respectively. The tumor volumes (V) were calculated using a caliper and the standard equation: V (mm³)=0.52 (ab²) (a=length, b=width). Once the tumor reached 100-200 mm³, the mice were randomly divided into different groups and subcutaneously injected with a vehicle or each peptide every day.

### 1-9. Measurement of Serum Testosterone Level

6-week-old male BALB/c mice (Jung Ang Lab Animal Inc., Seoul, South Korea) were subcutaneously injected with vehicle or 1 mg/kg/day HS1002 for 10 days. Blood samples were collected 5 h after initial injection and 5 h after last injection. The serum concentration of testosterone was determined using a testosterone ELISA kit (ALPCO Diagnostics, Salem, NH, USA).

### 1-10. Tumor Digestion and Flow Cytometry

A mouse tumor dissociation kit (Cat. 130-096-730, Miltenyi Biotec, Bergisch Gladbach, Germany) was used for tumor digestion according to the manufacturer's protocol. Briefly, the tumor was cut into small fragments with RPMI 1640 containing enzymes D, R, and A. After filtration through a 70 μm cell strainer, red blood cells were removed using RBC lysis buffer (Cat. 420301, BioLegend, San Diego, CA, USA). The acquired single-cell suspension was used for further analysis. For analysis of intracellular cytokines, the cell suspension was incubated in the presence of a cell stimulation cocktail (with a protein transport inhibitor) (Cat. 00-4975-93, Invitrogen, eBioscience) for 5 h and subjected to fixation and permeabilization using an intracellular fixation and permeabilization buffer set (Cat. 88-8824-00, Thermo Fisher) according to the manufacturer's protocol. Following the staining process, samples were analyzed with Novocyte (ACEA Biosciences, San Diego, CA, USA).

### 1-11. Library Preparation, Sequencing, and Data Analysis

Total RNA was isolated using QIAzol™ Lysis Reagent (Cat. 79306, Qiagen Sciences, MD, USA). RNA quality was examined by the TapeStation4000 System (Agilent Technologies, Amstelveen, The Netherlands), and RNA quantification was performed using the ND-2000 Spectrophotometer (Thermo Inc., DE, USA). Libraries were constructed from total RNA using the NEBNext Ultra II Directional RNA-Seq Kit (NEW ENGLAND BioLabs, Inc., UK). The isolation of mRNA was conducted using the Poly(A) RNA Selection Kit (LEXOGEN, Inc., Austria). cDNA synthesis and shearing were made from the isolated mRNAs, following the manufacturer's instructions. Indexing was conducted using the Illumina indexes 1-12. The enrichment step was performed using PCR. Subsequently, libraries were evaluated using the TapeStation HS D1000 Screen Tape (Agilent Technologies, Amstelveen, The Netherlands) to check the mean fragment size. Quantification was conducted using the library quantification kit using a StepOne Real-Time PCR System (Life Technologies, Inc., USA). High-throughput sequencing was conducted as paired-end 100 sequencing using NovaSeq 6000 (Illumina, Inc., USA). A quality control of raw sequencing data was conducted using FastQC. Adapter and low-quality reads (<Q20) were removed using FASTX_Trimmer and BBMap. Subsequently, the trimmed reads were aligned to the reference genome using TopHat. The read count data were analyzed based on the FPKM+Geometric normalization method using EdgeR within R. FPKM (Fragments Per kb per Million reads) values were calculated using Cufflinks. Data mining and graphic visualization were performed using ExDEGA (Ebiogen Inc., Korea).

### 1-12. Statistical Analysis

All the data are expressed as the mean±standard deviation (SD) of at least three independent experiments. The analysis of statistical significance was conducted using one-way analysis of variance (ANOVA), followed by Tukey's post-hoc comparisons test. Analysis was conducted using GraphPad Prism Software version 5.0 (GraphPad Software, CA, USA). A p value <0.05 indicated statistical significance.

### Example 2: Correlation of Prostate Cancer with hTERT Protein and Telomerase Activity

### 2-1. hTERT Protein Expression Pattern in Prostate Cancer Patients' Tissues

Human prostate cancer tissues and normal prostate tissues were purchased from Biomax, and the expression level of hTERT was observed by immunohistochemistry. First, paraffin was melted at 60° C. for paraffin removal. The tissues were treated with xylene for 10 min to completely remove paraffin and then sequentially treated with high-concentration alcohol to low-concentration alcohol to remove xylene. The tissues were blocked with a blocking solution, and 30 min later, hTERT antibodies (cell staining) were mixed at a ratio of 1:100 and allowed to bind at 4° C. overnight. The next day, secondary antibodies (1:1000) were allowed to bind, and then the ABC staining kit (staining kit) was used for 30 min. After the final DAB staining, the tissues were again dehydrogenated to complete staining.

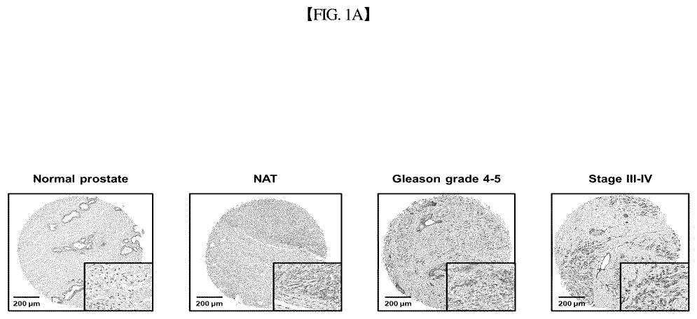

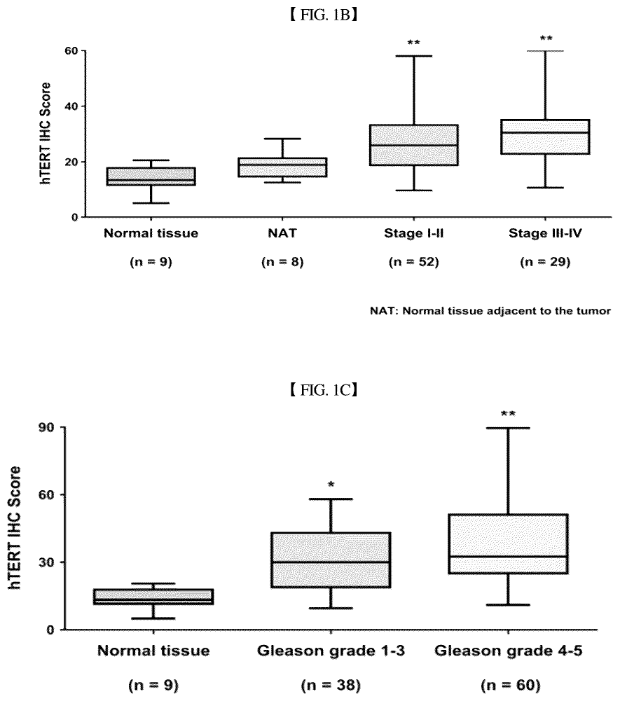

The results are shown in FIGS. 1A, 1B, and 1C.

As shown in FIG. 1A, as a result of measuring the expression of hTERT protein according to the progression of cancer by using immunohistochemistry (IHC) in tissues of human prostate cancer patients, strong staining was observed mainly in the nuclei of tumor cells. As shown in FIGS. 1B and 1C, the hTERT protein was over-expressed in the high-grade stages and Gleason grades compared with the normal tissue and normal tissue adjacent to the tumor (NAT). These results indicate that prostate cancer is correlated with hTERT protein expression.

### 2-2. hTERT Protein Expression and Telomerase Activity in Prostate Cancer Cell Lines

After prostate cancer cell lines were dissolved to isolate proteins, western blotting was performed using SDS-PAGE. The telomerase activity was analyzed using a telomerase activity quantification qPCR assay kit (ScienCell, Carlsbad, CA, USA).

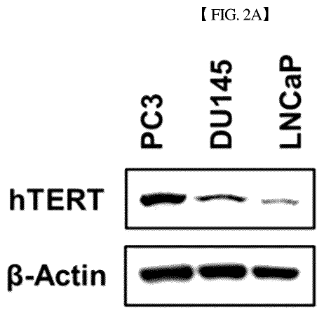

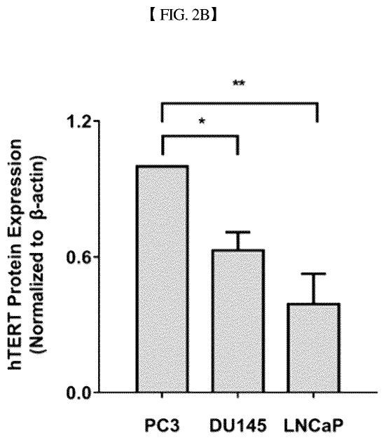

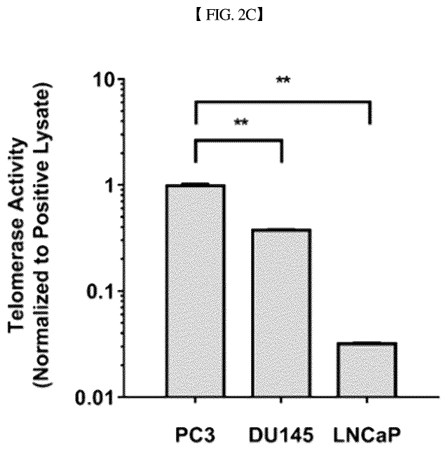

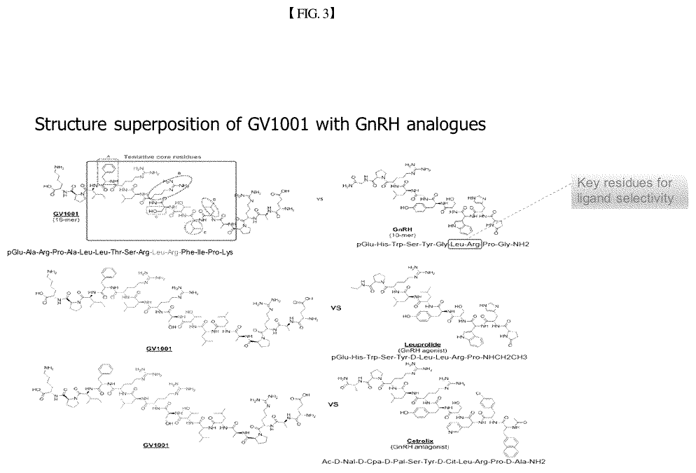

The results are shown in FIGS. 2A, 2B, and 2C.

As shown in FIGS. 2A, 2B, and 2C, as a result of investigating the hTERT expression levels in the prostate cancer cell lines (DU145, LNCaP, and PC3), PC3 showed the highest level of hTERT protein expression and the highest telomerase activity among the three types of cell lines. LNCaP with the lowest level of hTERT protein expression showed the lowest telomerase activity among the three types of cell lines. PC3 with the highest level of hTERT protein expression showed the highest telomerase activity among the three types of cell lines. These results indicate that the hTERT protein expression is correlated with the telomerase activity in prostate cancer cell lines.

### Example 3: Production of Peptide Targeting hTERT

The position of key amino acids of a peptide targeting telomerase was explored by comparing structural differences of a previously known gonadotropin-releasing hormone (GnRH) receptor ligand and a telomerase-derived peptide (GV1001).

The results are shown FIG. 3.

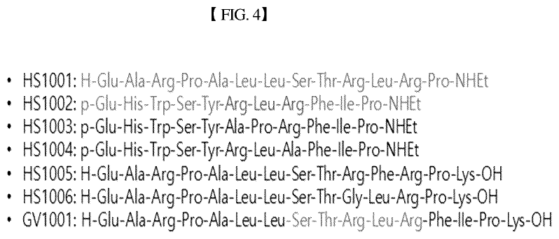

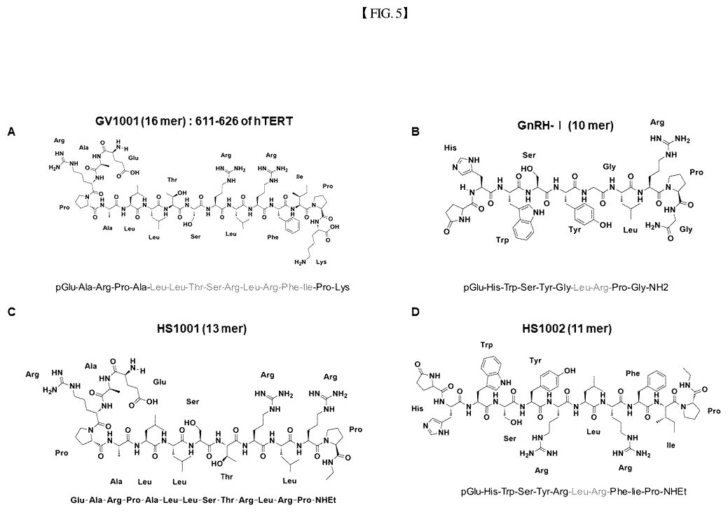

As shown in FIG. 3, as a result of verifying which amino acid sequence played a key role, Gly-Leu-Arg was predicted to be a core amino acid sequence. Six types of peptides (SEQ ID NO: 1 to SEQ ID NO: 6) were synthesized based on the sequence. The amino acid sequences and structures of the synthesized peptides are shown in FIGS. 4 and 5.

The peptide HS1002 was synthesized by coupling amino acids one by one from the C-terminus through Fmoc solid-phase peptide synthesis (SPPS) that has been conventionally known. The specific process for producing the peptide HS1002 is described as follows.

### 1) Resin Loading & Fmoc Deprotection

Fmoc-Ile-Trityl resin (0.77 mmole/g) was swelled in dimethylformamide (DMF) for 30 min and then washed two times with DMF. Thereafter, a 20% piperidine/DMF solution was added, followed by stirring for 10 min and subsequent draining. The above-described procedure was repeated two times, and the resin was washed six times with DMF.

### 2) Fmoc-A.A-OH Coupling

Fmoc-A.A-OH dissolved in DMF was placed in a reactor containing the resin, and then HBTU and N-methylmorpholine/DMF were added, followed by stirring for 1 h and subsequent draining. Thereafter, the resin was washed three times with DMF. Then, 20% piperidine/DMF was placed in the reactor containing the resin, followed by stirring and subsequent draining. The above-described procedure was repeated two times, and the resin was washed six times with DMF.

### 3) Repeating of Step 2) and Washing

The coupling of each amino acid was repeated as described in step 2). The 8-mer amino acid reaction was completed, and then the resin was washed three times with DMF.

### 4) Pyroglutamic Acid Coupling

Pyroglutamic acid (10 eq) dissolved in DMF was placed in the reactor containing the resin, and then HOBt (10 eq) and DIC (10 eq) were added, followed by coupling for 4 h and subsequent draining. After the final amino acids were stirred, the resin was washed three times with DMF and then washed three times with DCM, and then the resin was dried.

### 5) Resin Cleavage

MC/TFE/AcOH-8:1:1 were placed in the reactor containing the resin and stirred for 2 h, and then the filtered solution was collected in a conical tube. After additional stirring for 1 h, the filtered solution was collected in a conical tube. The solution was washed two times with H₂O, and then layer separation was performed. The DCM layer was removed using air, DCM was evaporated by nitrogen, and then freeze-drying was performed by addition of water and ACN.

### 6) Pro-NHEt·HCl Coupling

The peptide obtained in step 5) was dissolved in DMF, and then Pro-NHEt·HCl (10 eq) and HOAt (10 eq) were added, followed by cooling to 0° C. Thereafter, DIEA (10 eq) and EDC·HCl (10 eq) were added, followed by reaction for 12 h (reaction concentration: 0.2 M). Subsequently, it was investigated by HPLC and Mass whether reaction occurred. The reaction product was washed two times with H₂O, followed by layer separation, then the DCM layer was evaporated by air, and then freeze-drying was performed.

### 7) Global Cleavage)

The trifluoroacetic acid/1,2-ethanedithiol/thioanisole/H₂O (v:v:v:v-87.5%/2.5%/5.0%/5.0%) cleavage solution was added to the dried peptide, followed by stirring for 4 h. The cleavage solution was gently added to a cold ether to precipitate crystals. The crude peptide was dried in a vacuum dryer to give a crude.

### Example 4: Cytotoxicity of Produced Peptides to Cancer Cells

### 4-1. Cytotoxicity to Prostate Cancer Cell Lines

The prostate cancer cell line (LNCaP), in which the reactivity of HS1002 to hTERT had been best, were used to investigate the cytotoxic effects of GV1001, GnRH, HS1001, and HS1002 under conditions of 1,500 cells/well, 1% FBS, and 48-hour treatment. The prostate cancer cells were seeded at 1.5×10³ cells per well in a 96-well plate, and then incubated in an incubator for one day. Thereafter, the cells were treated with HS1001, HS1002, GV1001, and GnRH diluted to (300, 200, 100, and 50 μM) in fresh media. After 48-h incubation, an MTT reagent (3-(4,5-dimethylthiazol-2-yl)-2,5-diphenyltetrazolium bromide, Sigma-Aldrich, M6494) was added, and subsequently the plate was shielded from light and kept in an incubator for 3 h. Thereafter, the MTT solution was discarded, and 100 μL of dimethyl sulfoxide (DMSO, 99.9%, HPLC grade, DAEJUNG CHEMICALS & METALS, 3047-2304) was added to each well, and subsequently the plate was shielded from light and then left at room temperature for 20 min. After that, the absorbance was measured at 540 nm using a microplate reader.

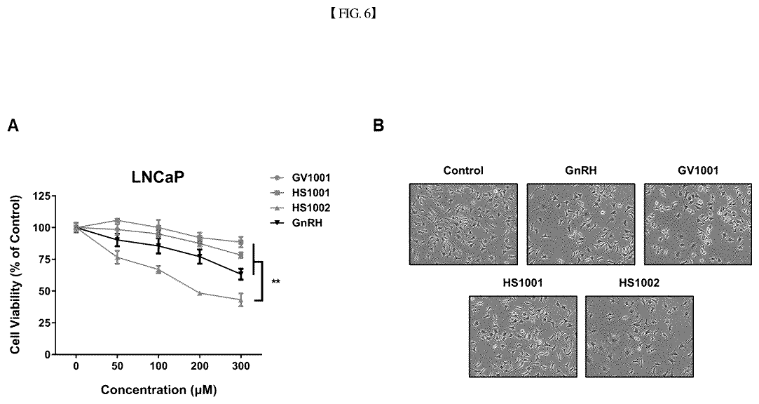

The results are shown FIG. 6.

As shown in FIG. 6, under treatment at the same concentration (300 μM), a microscopically distinct morphological change compared with the control group could be observed and the cytotoxicity to prostate cancer cells was highest, in the group treated with HS1002, among GV1001, GnRH, HS1001, and HS1002.

### 4-2. Apoptosis Promoting and Proliferation Inhibitory Effects on Prostate Cancer Cell Lines

It was investigated whether the treatment with the synthesized peptides had apoptosis promoting and proliferation inhibitory effects in prostate cancer cells. First, the prostate cancer cell line (LNCaP) was seeded into 6-well plates and then incubated in an incubator for one day. The peptides were diluted to 300 μM in fresh media and then used to treat the cells. After 72 h, the media were removed, and the plates were washed twice with DPBS, and 1 mL of trypsin was placed in each well to separate the cells, and 5 mL of media were added to dilute the trypsin, followed by centrifugation at 1000 rpm for 5 min, thereby preparing the cells. For the measurement of apoptosis, 1 mL of 1× binding buffer, 5 μL of annexin V, and 5 μL of PI were added to each sample by using an apoptosis kit, and the samples were shielded from light, kept at room temperature, and a flow cytometer was used.

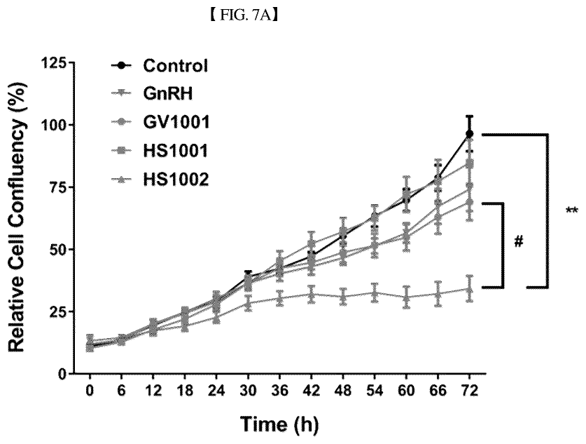

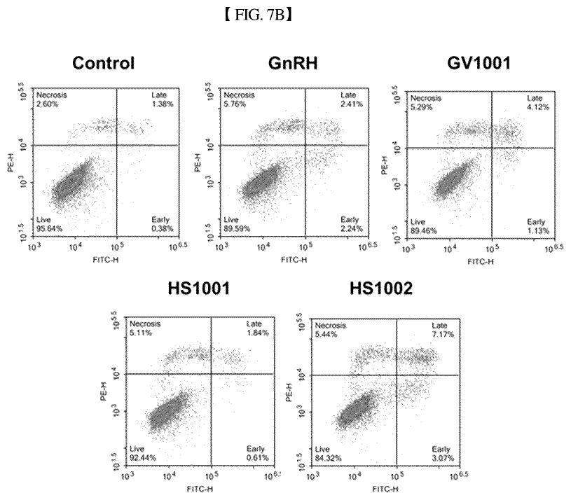

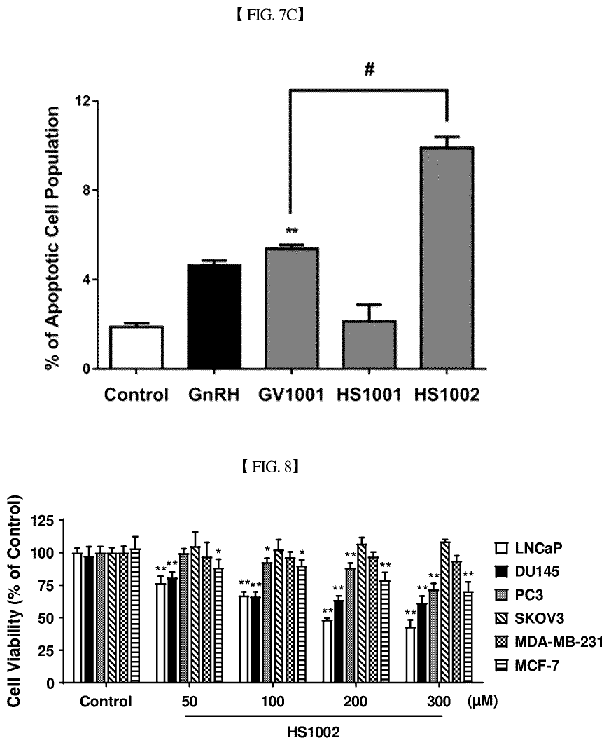

The results are shown in FIGS. 7A, 7B, and 7C.

As shown in FIG. 7A, as a result of investigating the cell proliferation for 72 h by using a real-time cell analysis system (InCucyte) after treatment of LNCaP cells with the peptides, HS1002 showed about 75% inhibition in the proliferation of prostate cancer cells (LNCaP) compared with the control group, and such an inhibition was superior other than the other treatment groups showing about 20% to 40% inhibition in cell proliferation. These results indicate that the peptide HS1002 of the present disclosure has an excellent proliferation inhibitory effect on prostate cancer.

### 4-3. Anticancer Effects on Various Types of Cancer Cells

To evaluate anticancer activities of the produced peptides on various types of cancer cells, MTT analysis was performed. Human prostate cancer cell lines (LNCaP, PC3, and DU145), human breast cancer cell lines (MDA-MB-231 and MCF-7), lung cancer cell line (A549), and ovarian cancer cell line (SKOV3) were cultured in RPMI media supplemented with 1% penicillin/streptomycin and 1% fetal bovine serum (all from Life Technologies, Grand Island, NY). The cells were seeded in 96-well plates containing 100 μL of growth media for 24 h. The media were removed, and then 100 μL of the peptide (solvent DPBS) at each concentration was added to each well and incubated at 37° C. for 48 h. After incubation for 48 h, 100 μL of MTT reagent was added to each well. The cells were incubated at 37° C. for 4 h, and then the supernatant was removed. Formazan crystals were dissolved in 100 μL of DMSO with gentle stirring at 37° C. for 10 min. The absorbance per well was measured at 540 nm using a VERSA max microplate reader (Molecular Devices Corp.).

The results are shown in FIG. 8 and Table 1.

As shown in FIG. 8 and Table 1, the peptide HS1002 showed excellent anticancer activity on the prostate cancer cell lines in the comparison on the types of cancer cells, especially showing excellent activity on LNCaP and DU145. These results indicate that HS1002 produced by the method of the present disclosure can be utilized as a novel inhibitor for prostate cancer.

TABLE 1
Cytotoxicity of produced peptides on various types of cancer cells

| | Cytotoxicity (IC₅₀ (μg/mL)) | | | | | |
|---|---|---|---|---|---|---|
| | | | | Lung | Breast | |
| | | | | cancer | cancer | Ovarian |
| Treatment | Prostate cancer cell line | | | cell line | cell line | cell line |
| group | LNCaP | PC3 | DU145 | A549 | MCF-7 | SKOV3 |
| GV1001 | 7.34 ± 0.31 | 9.05 ± 0.62 | 7.59 ± 0.46 | 3.16 ± 0.30 | 55.56 ± 2.18 | 5.16 ± 0.62 |
| HS1001 | 6.55 ± 0.96 | 8.05 ± 1.58 | 7.82 ± 0.37 | 4.18 ± 0.15 | 48.26 ± 1.23 | 6.14 ± 0.79 |
| (SEQ ID | | | | | | |
| NO: 1) | | | | | | |
| HS1002 | 6.83 ± 0.72 | 7.56 ± 0.61^{#} | 5.23 ± 0.65^{#} | 7.94 ± 0.07^{#} | 53.31 ± 1.79 | 4.91 ± 0.41^{#} |
| (SEQ ID | | | | | | |
| NO: 2) | | | | | | |
| HS1003 | 10.9 ± 0.48 | 24.05 ± 1.13 | 12.37 ± 0.31^{#} | 9.48 ± 0.08 | 47.28 ± 2.05 | 5.79 ± 0.38 |
| (SEQ ID | | | | | | |
| NO: 3) | | | | | | |
| HS1004 | 9.97 ± 0.85 | 16.49 ± 0.74 | 17.54 ± 0.74 | 5.14 ± 0.27^{#} | 37.47 ± 1.58 | 5.19 ± 0.82 |
| (SEQ ID | | | | | | |
| NO: 4) | | | | | | |
| HS1005 | 11.23 ± 1.01 | 13.31 ± 1.05 | 16.17 ± 1.28^{#} | 14.75 ± 0.83 | 44.15 ± 2.63 | 14.08 ± 1.04 |
| (SEQ ID | | | | | | |
| NO: 5) | | | | | | |
| HS1006 | 14.17 ± 0.47 | 15.2 ± 1.12 | 19.46 ± 0.26 | 10.68 ± 0.25 | 55.24 ± 2.41 | 13.91 ± 1.17 |
| (SEQ ID | | | | | | |
| NO: 6) | | | | | | |
| PTX | 0.81 ± 0.09 | 59.4 ± 3.21 | 0.73 ± 0.04 | 3.62 ± 0.04 | 9.27 ± 1.12 | 5.41 ± 0.19 |

PTX: Paclitaxel

### Example 5: Changes in hTERT Expression Level and Telomerase Activity by Treatment with Produced Peptides

### 5-1. Changes in hTERT Expression Level and Telomerase Activity

To analyze the changes in the hTERT expression level and telomerase activity by the treatment with the produced peptides, real-time PCR was performed. Total RNA was extracted from cells by using an Easy-blue RNA extraction reagent (Intron, Korea), and subsequently, the extracted RNA was reverse transcribed using M-ML V reverse transcriptase (Invitrogen, CA). The PCR reaction was then performed using a LightCycler 480 SYBR Green I Master (Roche, Germany) according to the manufacturer's instructions. Total RNA was extracted from cells by using a Qiazol reagent (Qiagen, Hilden, Germany), and 1-2 μg of RNA was reverse transcribed into cDNA by using a RevertiAid First Strand cDNA Synthesis kit (Thermo Scientific, Vilnius, Litheania). PCR was performed by a Biorad thermal cycler. The sequences of primers were as follows:

hTERT
(SEQ ID No: 7)
5′-TGA CAC CTC ACC TCA CCC AC-3′
(sense)
and
(SEQ ID No: 8)
5′-CAC TGT CTT CCG CAA GTT CAC-3′;
(antisense)
GAPDH
(SEQ ID No: 9)
5′- CGG AGT CAA CGG ATT TGG TCG TAT-3′
(sense)
and
(SEQ ID NO: 10)
5′- AGC CTT CTC CAT GGT GGT GAA GAC-3′.
(antisense)

Quantitative real-time RT-PCR (qRT-PCR) was performed using a Power SYBR Green 1-Step Kit and an ABI 7000 Real Time PCR System (Applied Biosystems, Carlsbad, CA, USA) according to the manufacturer's indications. The amplification protocol was composed of initial reverse transcription at 48° C. for 30 min, 40 cycles of denaturation at 95° C. for 15 s, and annealing and extension at 60ºC for 1 min. The results were expressed as the ratio of target PCR product to GAPDH product. The levels were normalized to glycer-aldehydes-3-phosphate dehydrogenase (GAPDH) levels. The quantification was performed using the ΔΔCT method (Applied Biosystems) with the RQ manager 1.2 software. Data are expressed as mean±SD. Each experiment was repeated three times for all samples.

The prostate cancer cell line (LNCaP) was seeded into six-well plates and then incubated in an incubator for one day. The peptides were diluted in fresh media and then used to treat the cells. After 48 h, the media were removed, the plates were washed twice with DPBS, and 1 mL of trypsin was placed in each well to separate the cells, and 5 mL of media were added to each well to dilute the trypsin, followed by centrifugation at 1000 rpm for 5 min, thereby preparing the cells. Thereafter, proteins were extracted using a Pro-prep protein extraction kit, separated by electrophoresis, and blotted onto a membrane. The membrane was blocked by a Blotting-Grade Blocker at room temperature for 1 hour, and then primary antibodies (1000:1) were added, followed by incubation at 4° C. for one day. Thereafter, the membrane was repeatedly washed with 1×TBST (Tris 25 g, NaCl 120 g in D.W.+Tween 200 0.1%) every 10 min for 1 h. Then, secondary antibodies (10000:1) were added, followed by incubation at room temperature for 1 h, and then the membrane was repeatedly washed with 1×TBST every 10 min for 1 h. After incubation with HRB substrates for 5 min, desired protein bands were identified using a Chemi-doc system.

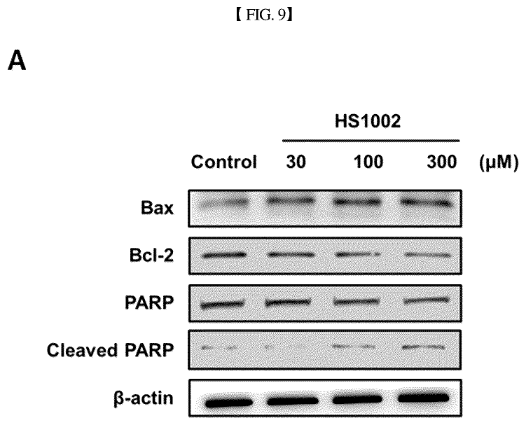

The results are shown in FIG. 9.

In the treatment with HS1002, the expression levels of BAX and C-PARP, proteins involved in apoptosis, were increased and the expression level of Bcl-2 was decreased, in a concentration-dependent manner. These results indicate that the peptide HS1002 had an effect of inducing apoptosis in prostate cancer cells.

After treatment with GV1001, HS1001, HS1002, and GnRH, the changes in hTERT mRNA and protein expression level were investigated for each peptide. The drug treatment was performed every 24 h considering the stability of peptides in aqueous solutions. To investigate the telomerase activity reducing effect of the peptides, prostate cancer cell lines (LNCaP) were treated with each drug at 300 μM every 24 h for a total of 72 h and then measured for telomerase activity through qPCR. The proteins in a cell lysate were quantified to obtain a telomerase product extended by the same amount of proteins, and qPCR was performed using the telomerase as a template.

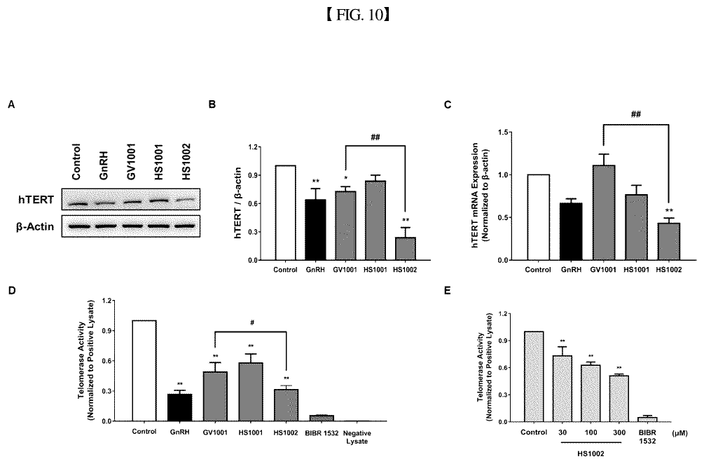

The results are shown in A, B, and C of FIG. 10.

As shown in A, B, and C of FIG. 10, western blot and qPCR identified that the hTERT expression was significantly reduced in the HS1002-treated group compared with the control, GV1001, and HS1001. Compared with the control group, the hTERT expression was reduced by about 10 to 30% in the GV1001- and HS1001-treated groups, while the hTERT expression was reduced by about 50 to 80% in the HS1002-treated group.

In the treatment with the peptides, the change in telomerase activity was investigated. The cells were cultured in 100-mm plates each, and about 24 h later, the cells were treated with the peptides synthesized in Example 3 at a concentration of 300 μM before incubation. The cells were cultured in a 37° C., 5% CO₂ incubator using 1% FBS RPMI media. The cells were harvested 72 h after the peptide treatment and measured for telomerase activity. A telomerase activity quantification qPCT analysis kit (ScienCell, Carlsbad, CA, USA) was used.

The results are shown in D and E of FIG. 10.

As shown in D and E of FIG. 10, as a result of analyzing telomerase activity, the hTERT expression and telomerase activity in prostate cancer cells (LNCaP) were reduced by HS1002 treatment. Compared with the control group, the telomerase activity was reduced by about 30 to 50% in the GV1001- and HS1001-treated groups, while the telomerase activity was reduced by about 50 to 70% in the HS1002-treated group.

The cells were treated with HS1002, showing a better effect than GV1001 in the cell experiments, at different concentrations, and then the concentration-dependent reduction in hTERT protein was investigated by western blot.

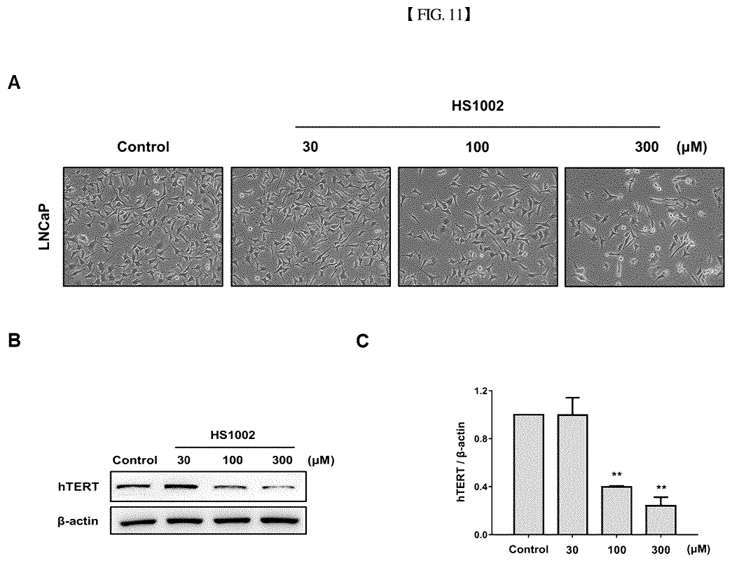

The results are shown in FIG. 11.

As shown in FIG. 11, the hTERT protein expression was reduced with an increasing concentration of HS1002, and HS1002 showed better telomerase activity than GV1001.

### 5-2. HS1002 Regulates hTERT Expression Through the Various Signaling Pathway

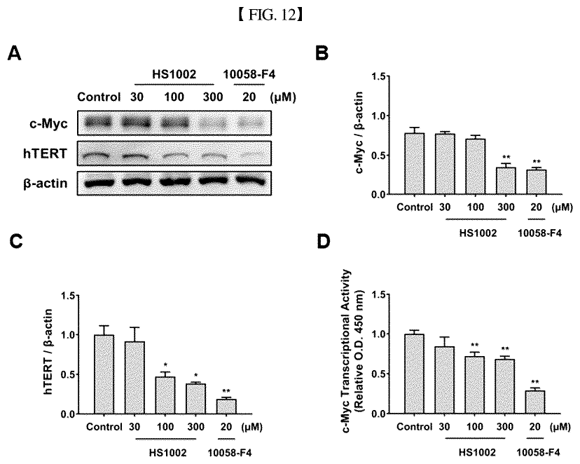

To determine the mechanism by which HS1002 regulates hTERT and telomerase activity, the expression of transcription factors and their upstream signaling proteins essential for hTERT regulation was confirmed. To confirm the connection between HS1002 and c-Myc activity, the transcription activity of c-Myc and its protein expression were measured after treatment with HS1002 or the c-Myc inhibitor (10058-F4). Both c-Myc protein expression and transcriptional activity were significantly decreased in LNCaP cells treated with 300 μM HS1002 (FIG. 12).

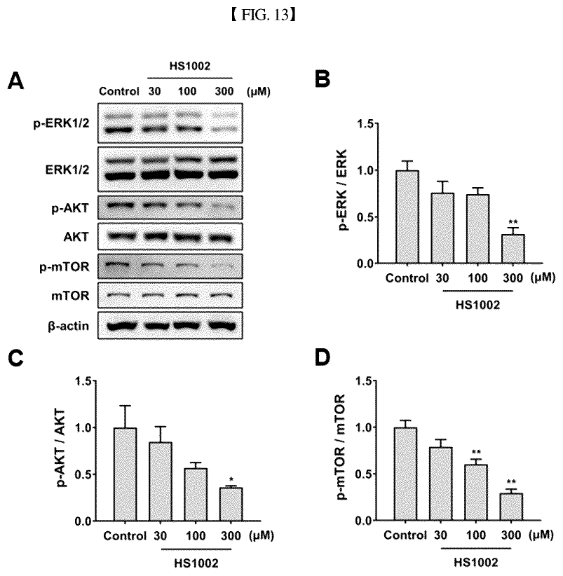

Furthermore, a western blot analysis assessed the expression levels of signaling pathway proteins that regulate hTERT in LNCaP cells treated with HS1002. These results demonstrated that the AKT, ERK1/2, and mTOR pathways were markedly inhibited in HS1002-treated LNCaP cells. HS1002 significantly downregulated the expression of p-AKT, p-ERK1/2, and p-mTOR. In addition, the expression of AKT, ERK1/2, and mTOR was unchanged in both the control and HS1002 groups (FIG. 13). These results indicate that HS1002 treatment resulted in a decrease in c-Myc activity and phosphorylation of signaling proteins, causing decreased hTERT expression and activity.

### 5-3. HS1002 Reduced the hTERT Expression in LNCaP Tumor Tissues

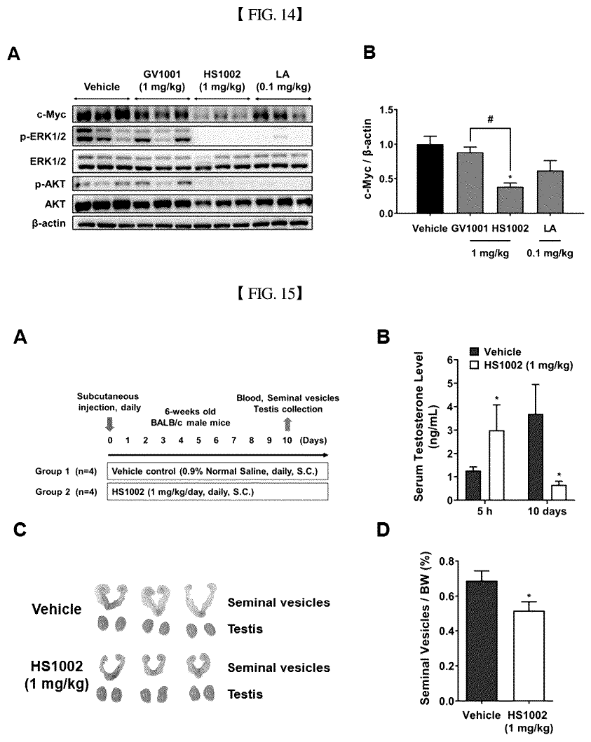

In a similar in vitro study, the protein expression of c-Myc, AKT, and ERK1/2 was examined in HS1002 tumor tissue. HS1002 and LA treatment significantly downregulated the expression of c-Myc, p-AKT, and p-ERK1/2. Interestingly, HS1002 decreased c-Myc expression more than LA (FIG. 14). Taken together, these results indicate that HS1002 treatment significantly suppresses hTERT expression, which might be closely related to the downregulation of c-Myc, AKT, and ERK in LNCaP tumor xenografts.

### Example 6: Action of Peptide on GnRH Receptor

GnRH agonists stimulate the release of LH and FSH by binding to GnRHR located on the pituitary gonadotropes and consequently stimulate the secretion of steroid hormone from the gonads. However, continuous exposure to the GnRH agonist results in the downregulation of desensitization of GnRHR and eventually decreases production of testosterone. The inhibitory effect on the pituitary gonadal axis has a role in the anti-prostate cancer effects of GnRH analogs. Based on the HS1002 peptide design strategy, to validate whether HS1002 functions as a GnRHR ligand, the serum testosterone level in male mice was tested after repeated subcutaneous injections of HS1002. Serum testosterone level was 2.37-fold increased 5 h after the 1st HS1002 injection compared to the vehicle-injected group. However, serum testosterone level at 10 days after repeated injection of HS1002 was significantly lower than in the vehicle-injected group. Moreover, seminal vesicle weights in HS1002-treated mice were significantly reduced compared to those in the vehicle-treated group (FIG. 15). The data indicate that continuous exposure to HS1002 may affect the function of the pituitary gonadal axis and inhibit the secretion of testosterone.

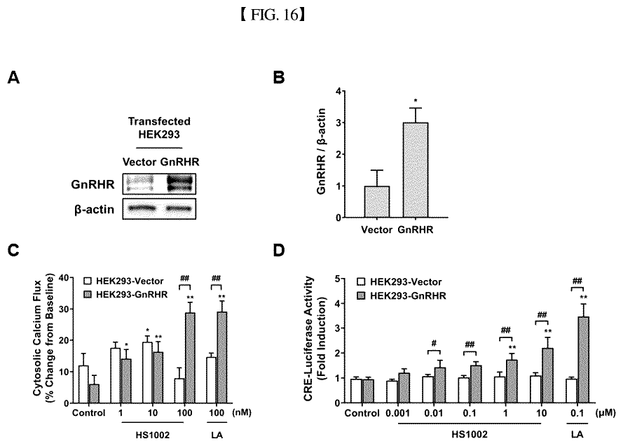

To confirm whether HS1002 induces GnRHR downstream signaling, a GnRHR-overexpressing HEK293 cell line was constructed. The western blot analysis indicated that the protein levels of GnRHR in HEK293-GnRHR cells were elevated compared to those in mock-transfected HEK293-Vectors. To investigate which downstream signaling pathway of GnRHR is activated by HS1002, Gaq-mediated intracellular calcium release and Gas-stimulated cAMP activity were examined after exposing HEK293-GnRHR cells to HS1002 and LA. 100 nM LA induced a more potent effect of calcium flux in HEK293-GnRHR cells than in HEK293-Vector cells. HS1002 (1 and 10 nM) increased calcium flux in both HEK293-GnRHR and HEK293-Vectors cells, but only HS1002 (100 nM) increased calcium flux in HEK293-GnRHR cells. As with the calcium flux assay result, 100 nM LA induced CRE-derived reporter gene activity in HEK293-GnRHR cells. There is no significant change in CRE-luciferase activity in HEK293-Vector cells. Interestingly, cAMP-mediated promoter binding activity was concentration-dependently enhanced by HS1002. At 10 μM, HS1002 increased the 2-fold CRE-luciferase activity compared to the control in HEK293-GnRHR cells (FIG. 16).

### Example 7: Anticancer Effect of Peptide in Animal Model

### 7-1. Preparation of Xenograft Animal Models

To investigate the anticancer effect of HS1002, the present inventors observed the volume of cancer cells after transplanting the cancer cells into experimental animals. Specifically, mice (4-week-old male BALB/c nude mice; Central Lab. Animal Inc., Seoul, Korea) were prepared, and tumor cells (1×10⁷ LNCaP cells) suspended in 1 mL of a serum-free medium containing 50% Matrigel were percutaneously injected into the upper flank of each nude mouse. When the tumor cell volume reached about 200 mm³, the mice were randomly assigned to a vehicle control group, a leuprolide acetate-treated group, and test groups (GV1001-treated group and HS1002-treated group), respectively. Leuprolide acetate (0.1 mg/kg/day) and GV1001 and HS1002 (1 mg/kg/day) were injected subcutaneously for 13 days.

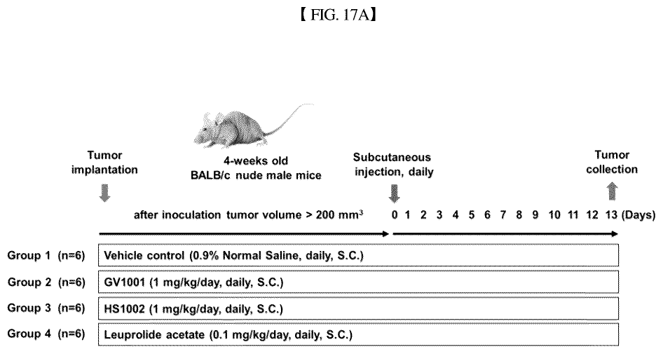

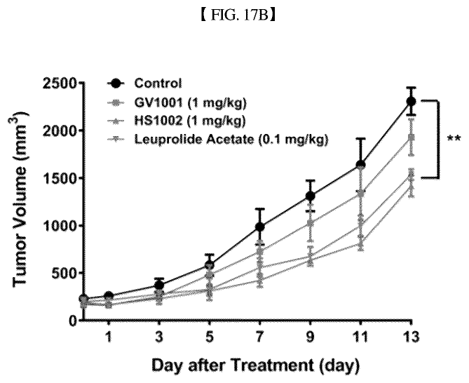

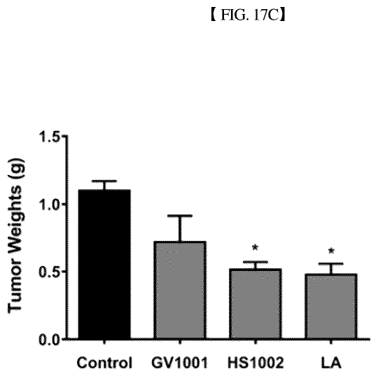

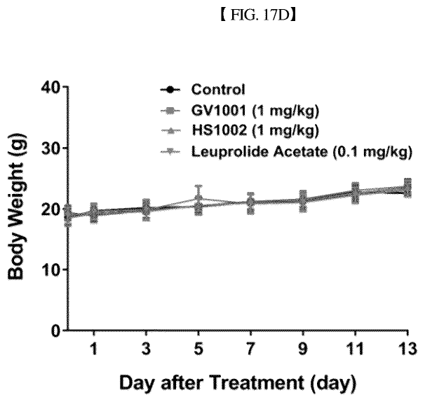

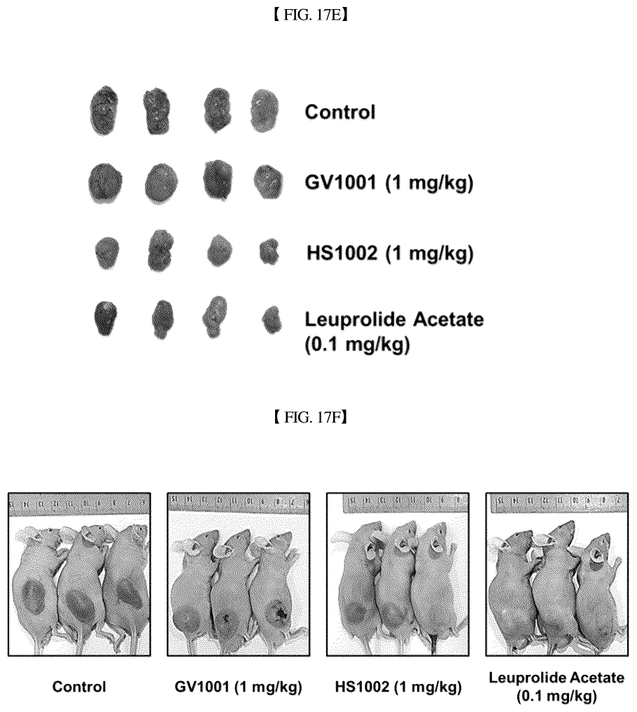

The results are shown in FIGS. 17A, 17B, 17C, 17D, 17E, and 17F.

As shown in FIG. 17D, the subcutaneous administration of HS1002 showed no change in body weight and no other toxicity. However, as shown in FIGS. 17B and 17C, the tumor volume and weight were reduced by about 50%. These results indicate that HS1002 has no or fewer side effects.

### 7-2. Changes in Ki-67 and hTERT Protein Levels in Tumor Tissue by Peptide Treatment

Mice were sacrificed, and the excised tumors were fixed in 10% formalin and impregnated with paraffin. For pathological examination, immunohistochemistry was performed by the avidin-biotin complex method using Ki-67 antibodies. The immune response was visualized with a reagent (3,3-diaminobenzidine) and counter-stained with Mayer's hematoxylin. TUNEL assay in tissue was performed using ApopTag Plus Peroxidase in Situ Apoptosis Detection kits (Intergen, Purchase, NY)) according to the manufacturer's indications. In summary, paraffin was removed from slides, and the slides were placed in 3% hydrogen peroxide to inhibit endogenous peroxidase. The slides were incubated in a reaction buffer containing an enzyme (terminal deoxynucleotidyl transferase) at 37° C. for 1 h. The slides were incubated with peroxidase-conjugated anti-digoxigenin antibodies for 30 min, and the reaction product was visualized with a solution containing 2 mmol hydrogen peroxide (0.03% 3,3-diaminobenzidine solution). Counterstaining was performed with 0.5% methyl green. Ki-67- and TUNEL-positive cells were counted and expressed as an average of the five highest areas within a single×200 field. A portion of tumor tissue was homogenized, followed by immunoblotting for hTERT.

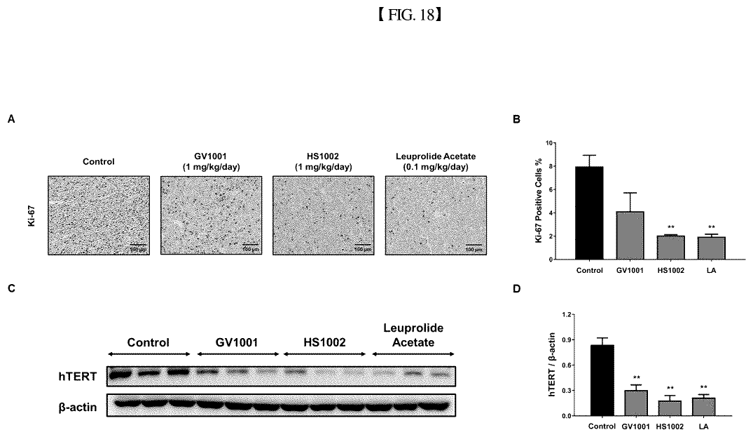

The results are shown in FIG. 18.

As shown in FIG. 18, the Ki-67 and hTERT protein expression levels were distinctively reduced in the tumor tissues of mice subcutaneously administered HS1002 compared with the control group. Since the Ki-67 protein is a biomarker capable of verifying the degree of proliferation of cancer cells, HS1002 was verified to also have an anticancer effect in in vivo models.

### 7-3. Anti-Cancer Effect of HS1002 in MC38 and 4T1 Syngeneic Mouse Model

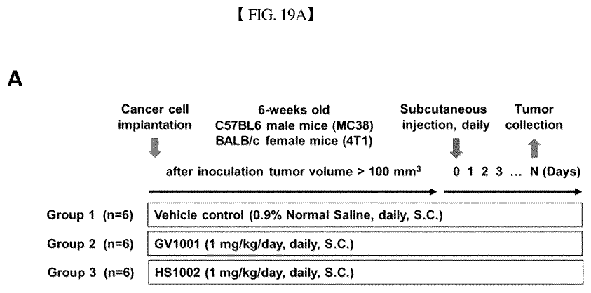

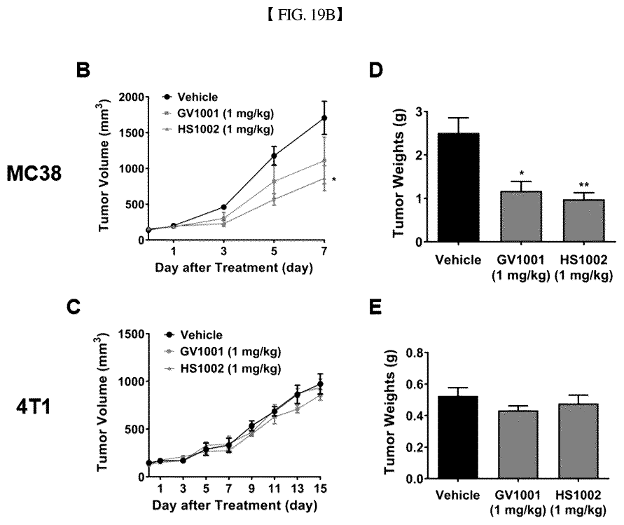

The sequences of the TERT peptide are highly conserved in human and mouse, indicating its potential translatability to mouse cancer. Syngeneic mouse model systems that possess a functional immune system can be employed for the purpose of investigating immunotherapies. ‘Hot’ tumors, such as the MC38 tumor model, are those with a high degree of T cell and CTL infiltration, as well as expression of the interferon signature. Hot tumors are generally more responsive to immunotherapy. In contrast, ‘Cold’ tumors such as the 4T1 tumor model lack infiltrating T cells and often have a low tumor mutational burden. Without a pre-existing adaptive immune response, cold tumors are insensitive to immunotherapy. To confirm the effect of HS1002 on anti-cancer immunity in different syngeneic mouse models, tumor growth inhibition was examined in the MC38 and 4T1 syngeneic mouse models (FIG. 19A). Interestingly, the tumor volume and weight were diminished by the administration of HS1002 or GV1001 in the MC38 but not in the 4T1 syngeneic mouse model (FIG. 19B). These results indicate that tumor immune microenvironment phenotypes might affect the anticancer effect of HS1002 in an in vivo syngeneic mouse model.

### 7-4 HS1002 Induces Anti-Tumor Immunity in MC38 Syngeneic Mouse Model

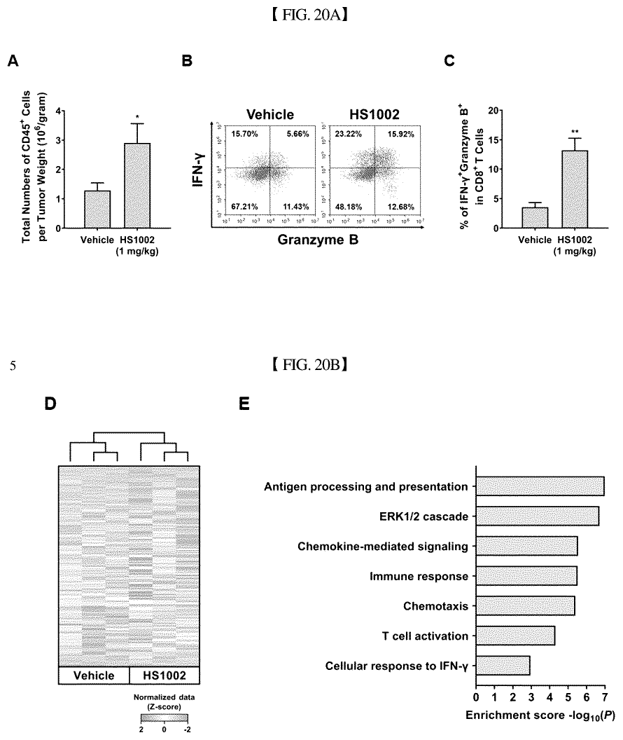

CTLs are the key mediators of granzyme- and perforin-mediated cancer cell destruction during cancer immunosurveillance and immunotherapy. IFN-γ also boosts the antitumor activities of other immune cells, suggesting that modulating CD8⁺ T cell activity can enhance antitumor immunity. To investigate the effect of HS1002 on tumor-infiltrating CD8⁺ T cells in MC38 tumor tissue, flow cytometric analysis was used. Flow cytometric analysis confirmed that HS1002 elevated the total number of tumor-infiltrating CD45+ cells and the production of granzyme B and IFN-γ in CD8⁺ cells and in MC38 tumor tissue (FIG. 20A). To assess the effect of HS1002 on immune response-related gene expression in a syngeneic mouse model, gene expression analysis was conducted by profiling the transcriptomes of the tumor. A total of 49,196 genes were analyzed in tumor tissues, and genes with a fold change exceeding 1.5 were included for further analysis with statistical significance at p<0.05. A total of 160 genes were significantly upregulated in HS1002-treated tumor tissue, whereas the expression of 69 genes was significantly downregulated in HS1002-treated tumor tissue. A one-way hierarchical clustering analysis of significant differentially expressed genes (DEG) showed that the HS1002-treated group displayed a prominent change in DEG relative to the vehicle group. Further gene ontology function analysis of these genes demonstrated a substantial association with immune response functions such as antigen processing and presentation, the ERK pathway, chemokine-mediated signaling, chemotaxis, T cell activation, and cellular response to IFN-γ (FIG. 20B). These data suggested HS1002 could induce anti-tumor immunity by increasing functional immune cell infiltration and cytotoxicity.

What is claimed is:

1. A polypeptide comprising the amino acid sequence of SEQ ID NO: 2.

2. A nucleic acid molecule comprising a nucleotide sequence encoding the polypeptide of claim 1.

3. A recombinant vector comprising the nucleic acid molecule of claim 2.

4. A host cell transformed with the recombinant vector of claim 3.

5. A pharmaceutical composition comprising a polypeptide comprising the amino acid sequence of SEQ ID NO: 2.

6. A method for preventing or treating cancer, the method comprising administering to a subject a pharmaceutical composition comprising a polypeptide comprising the amino acid sequence of SEQ ID NO: 2.

7. The method of claim 6, wherein the cancer is associated with an increase in telomerase reverse transcriptase expression, an increase in telomerase activity, or a combination thereof.

8. The method of claim 6, wherein the cancer is prostate cancer, ovarian cancer, or a combination thereof.


SEQUENCE LISTING (10 sequences)

SEQ ID NO: 1 - protein, 13 aa, synthetic construct
EARPALLSTRLRP

SEQ ID NO: 2 - protein, 11 aa, synthetic construct
EHWSYRLRFIP

SEQ ID NO: 3 - protein, 11 aa, synthetic construct
EHWSYAPRFIP

SEQ ID NO: 4 - protein, 11 aa, synthetic construct
EHWSYRLAFIP

SEQ ID NO: 5 - protein, 14 aa, synthetic construct
EARPALLSTRFRPK

SEQ ID NO: 6 - protein, 14 aa, synthetic construct
EARPALLSTGLRPK

SEQ ID NO: 7 - DNA, 20 bp, synthetic construct
  source 1..20: mol_type = other DNA
tgacacctcacctcacccac

SEQ ID NO: 8 - DNA, 21 bp, synthetic construct
  source 1..21: mol_type = other DNA
cactgtcttccgcaagttcac

SEQ ID NO: 9 - DNA, 24 bp, synthetic construct
  source 1..24: mol_type = other DNA
cggagtcaacggatttggtcgtat

SEQ ID NO: 10 - DNA, 24 bp, synthetic construct
  source 1..24: mol_type = other DNA
agccttctccatggtggtgaagac